(19) United States
(12) Patent Application Publication
(10) Pub. No.: US 2005/0015035 A1
(43) Pub. Date: Jan. 20, 2005

(54) TMC brace

(76) Inventors: Joseph Anthony Sansone, Houston, TX (US); Gordon Ryan Stelck, Houston, TX (US)

Correspondence Address: Attn: Joseph A. Sansone;TMC Orthopedic, Suite 150, 1000 South Loop West, Houston, TX, 77054, US

(21) Appl. No.: 10/769,608

(22) Filed: Oct. 4, 2004

Related U.S. Application Data
(60) Provisional application No. 60/444,801, filed on Feb. 4, 2003.

(51) Int. Cl. A61F 5/00

(52) U.S. Cl. 602/19

(57) ABSTRACT

Lumbo Sacral Orthosis (apparatus) is comprised of the anterior and posterior rigid medical grade ABS heat moldable plastic. The anterior and posterior panels are lined with foam covered with a Velcro hook and loop fastener type material. These are affixed to the panels by a hook and loop fastener. The anterior and posterior panels are placed on the torso of the patient with an adjustable strap system consisting of elastic material with an internal plastic moldable insert, which is attached to the posterior panel via two brass oxide rivets. The strapping system is attached to the anterior panel via hook and loop material. This allows for the patient to apply desired tension. The secondary strapping system is affixed to the posterior panel via two brass oxide rivets attached to heat moldable plastic material. The anterior panel secondary strapping system is affixed to the panel via two stainless steel rivets. The anterior and posterior secondary strapping system is then connected via a buckle system. The C-Fold from the posterior section is hook and loop material and is able to remove any pressure from the posterior section and allows the patient to apply additional tension.

This is a continuation application following the provisional application filed on Feb. 4, 2003, application No. 60444801. Below is the declaration of product claim and description.

The TMC brace . . .

Is a lumbar sacrial orthosis; lumbar spinal support.

The body of the brace consists of an anterior and a posterior medical grade velcro laminate foam which is removable from the rigid portions of the brace.

These separate liners may be hand-washed w/mild detergent and air-dryed. As these liners are separate from the rigid aspects of the brace, they may be replaced after excessive wear without the patient having to replace or purchase an entire new device.

Attached to the antior liner is a ⅛″ rigid, medical grade abs, heat moldable plastic which covers more than ⅓ of the entire liner in width and the full lenth of the liner in height, normally fitting the patient just below the asis to just below the patient's xyphoid process. This rigid plastic body is secured to the liner w/velcro hook coins allowing them to be removed for washing care of the liner.

On each side of the main rigid body are underlapping semi-rigid tongues which extend to each farthest end of the anterior liner.

These tongues are made from a medical grade, modified polyethylene plastic which is covered by velcro loop sewn to the main body of the anterior liner.

There are two anterior compression straps attached to each side of the main anterior plastic body and are made from 1½″ double-sided velcro loop and are attached to the main anterior rigid body with solid brass oxide-finished rivets to prevent corrosion and skin irritation of the patient.

These straps loop through the female portion of our unique “quick release” buckles and are secured back to the anterior plastic body.

Attached to the posterior liner is also a ⅛″ rigid medical grade abs thermo moldable main body plastic shell which is the same as the in width and all in height covering the patient from just above the glutial fold to the inferior scapula. The main posterior plastic body is attached to the liner w/velcro hook coins to make it removable from the liner for washing care as well.

On each side of the main post shell are very unique side panel straps which underlay the main posterior plastic shell. these side panel straps are connected to the posterior and anterior sections of each side and have enclosed medical grade polyethylene plastic inserts the entire length of the side panel strap, thus providing total circumerencial rigidity throughout the entire brace.

There are two 1½″ c-fold posterior compression straps connected to each side of the main posterior panel at the top and bottom into one 1½″ c-fold strap that is conecting the mail end of our “quick release” buckle system. All of these straps are secured w/solid brass oxide medical grade rivets to prevent corrosion and skin irrition to the patient.

Once the male and female “quick release” buckles are connected on each side of the brace, the anterior compression strap is pulled away from the patient through the loop on the quick release buckle forcing both anterior and posterior sections together creating compression.

When the patient wants to remove the brace, they simply squeeze the quick release buckle in the middle separating the anterior and posterior compression straps. then the plastic side panel strap is un velcroed from yhe anterior portion of the brace, usually on patient's dominate side. When the patient desires to re-don the brace, they simply reattach the elastic/loop/plastic insert side panel straps back to the anterior and “click” the “quick release” buckles back into place.

### BACKGROUND TMC BRACE

This device is a blend of a soft elastic corset and a more traditional rigid lumbo sacral orthosis. The apparatus which is modular in design allowing for multiple adjustments as well as a fit to a wide variety of patients' sizes due to its conformity, and its soft and flexible nature. The orthosis provides the compression and support of a rigid lumbo sacral orthosis and the comfort liner system increases patient compliance therefore the patient will wear the orthosis and limit the potential for further injury.

### SUMMARY OF INVENTION

The apparatus is a Lumbar Sacral Orthosis; Lumbar Spinal Support.

The body of the brace consists of anterior and posterior medical grade Velcro laminate foam which is removable from the rigid portions of the brace.

Attached to the anterior liner is a ⅛″ rigid, medical grade ABS, heat moldable plastic which covers more than one-third of the entire liner in width and full length of the liner in height, normally fitting the patient just above the symphis pubis to just below the patients Xyphoid process. This rigid plastic body is secured to the liner with Velcro hook coins allowing them to be removed for washing care of the liner.

On each side of the anterior rigid panel are under-lapping, semi-rigid tongues which extend to each farthest end of the anterior liner. These tongues are made from a medical grade, modified polyethylene plastic which is covered by a Velcro loop sewn to the main body of the anterior liner.

There are two anterior compression straps attached to each side of the main anterior plastic body and are made from 1½″ double sided Velcro loop and are attached to the main anterior rigid panel with solid brass oxide finished rivets. These straps loop through the male portion of our unique “Quick Release” buckles and are secured back to the anterior plastic body.

Attached to the posterior liner is also a ⅛″ rigid medical grade ABS heat-moldable main body plastic shell, which covers one-third of the posterior liner in width and all in height covering the patient from just above the Glutial fold to the inferior scapula. The main posterior plastic body is attached to the liner with Velcro hook coins.

On the left and right side of the main posterior shell are side panel straps which underlay the main posterior plastic shell. The side panel straps are attached to the posterior liner via hook and loop material. The side panel strap is made from hook and loop material sewn to elastic and has enclosed medical grade polyethylene plastic inserts ¾'s of the length of the side panel strap providing adjustability and total circumferential compression. The side panel straps connect the posterior and anterior sections of the LSO.

There are two, 1½″ c-fold posterior compression straps connected to the left and right side of the main posterior panel affixed to the top and bottom of the panel via brass oxide rivets the two straps are then connected to a single strap via a rivet the 1½″ c-fold strap is connecting the female end of our “Quick Release” buckle system. The posterior strap is attached to the anterior panel; the male connection is attached to female connection therefore attaching the posterior section. Once the male and female “Quick Release” buckles are connected on each side of the brace, the anterior compression strap is pulled away from the patient through the loop on the “Quick Release” buckle forcing both anterior and posterior sections together creating hydrostatic lift therefore providing the needed immobilization of the lumbo sacral spine compression.

When the patient wants to remove the brace, they simply squeeze either the left or right sides “Quick Release” buckle separating the anterior and posterior compression straps. Then the plastic side panel strap is un-velcroed from the anterior portion of the brace. When the patient desires to re-don the brace, they simple reattach the elastic/loop/plastic insert side panel straps back to the anterior and “Click” the “Quick Release” buckles back into place.

### BRIEF DESCRIPTION OF THE DRAWINGS

FIG. 1 shows the posterior rigid panel and liner, compression straps with female buckles and side panel straps.

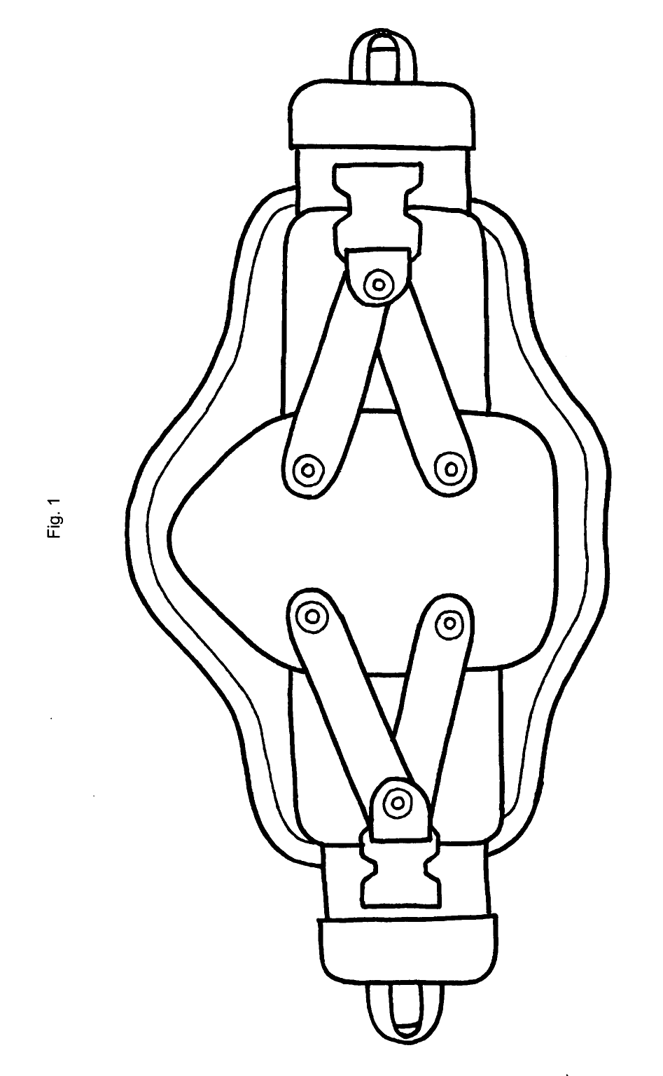

FIG. 2 shows the anterior rigid panel and liner, overlapping tongues and compression straps with male buckles.

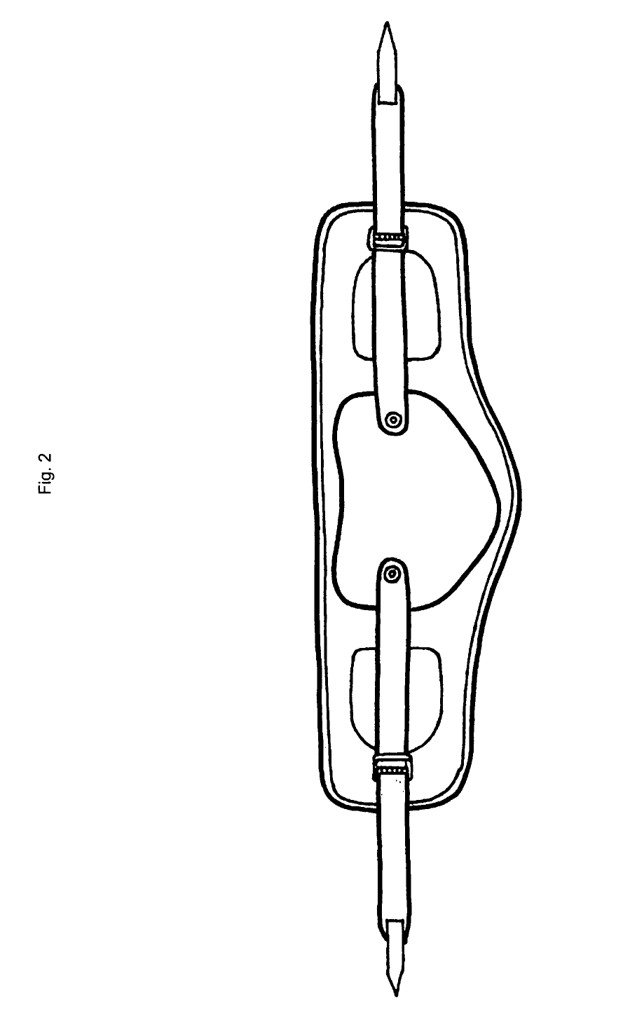

FIG. 3 frontal and reverse view of anterior compression strap with male buckle.

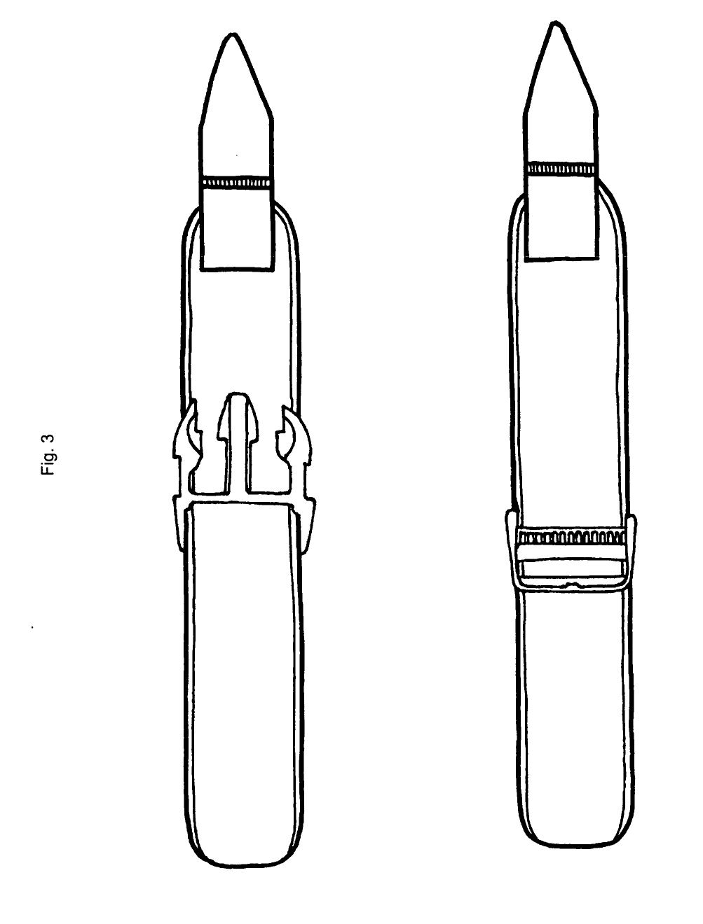

FIG. 4 posterior compression strap with “Y” and female buckle.

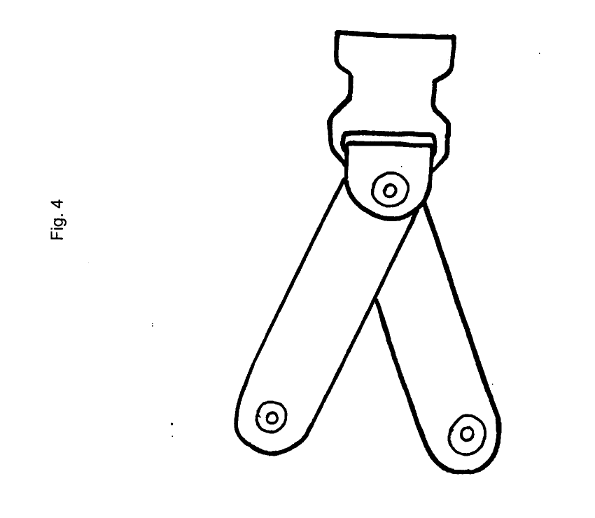

FIG. 5 compression strap with hook and loop, plastic insert, elastic panel and thumb loop.

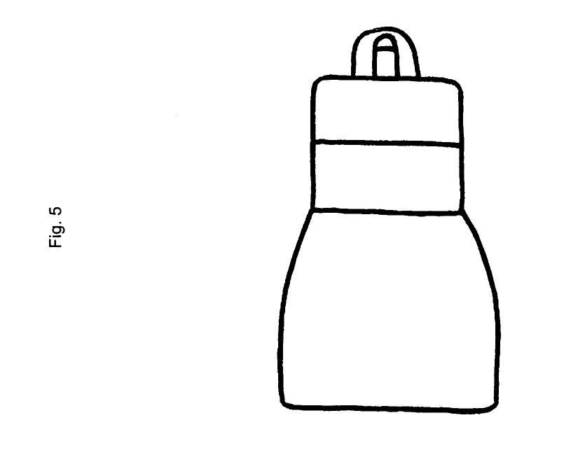

FIG. 6 removable posterior liner with surged edges.

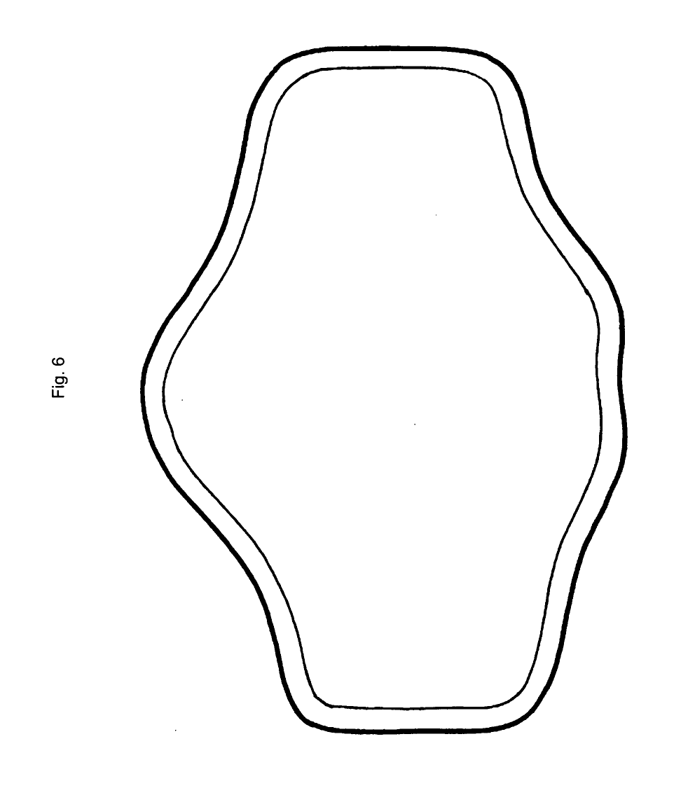

FIG. 7 removable anterior liner with overlapping tongues and surged edges.

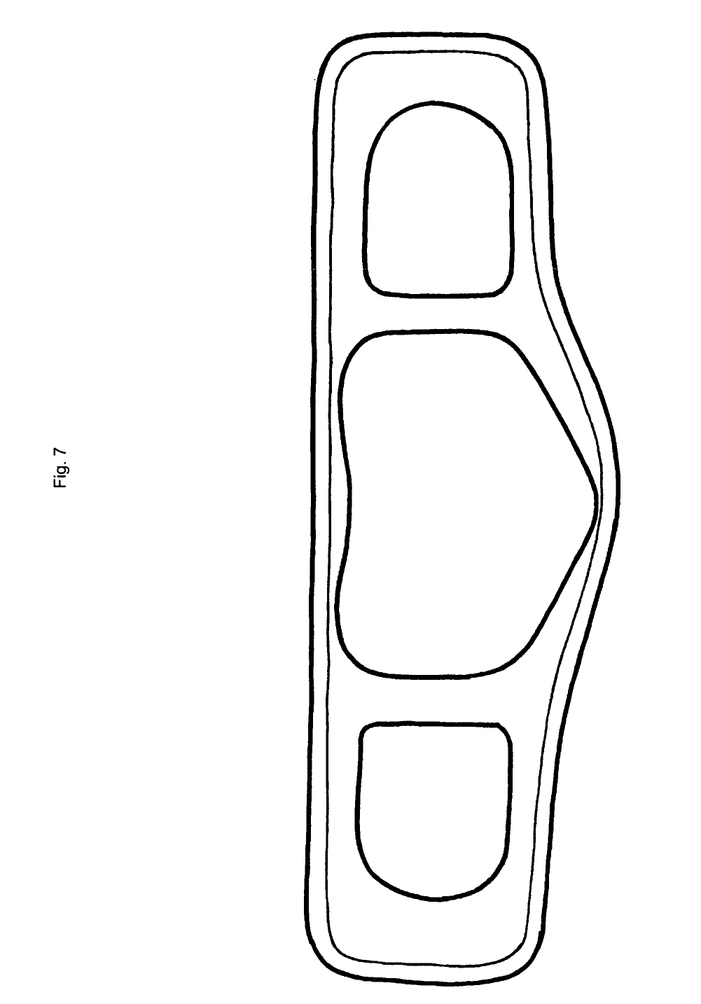

FIG. 8 posterior view of the rigid panel and liner with side panel straps.

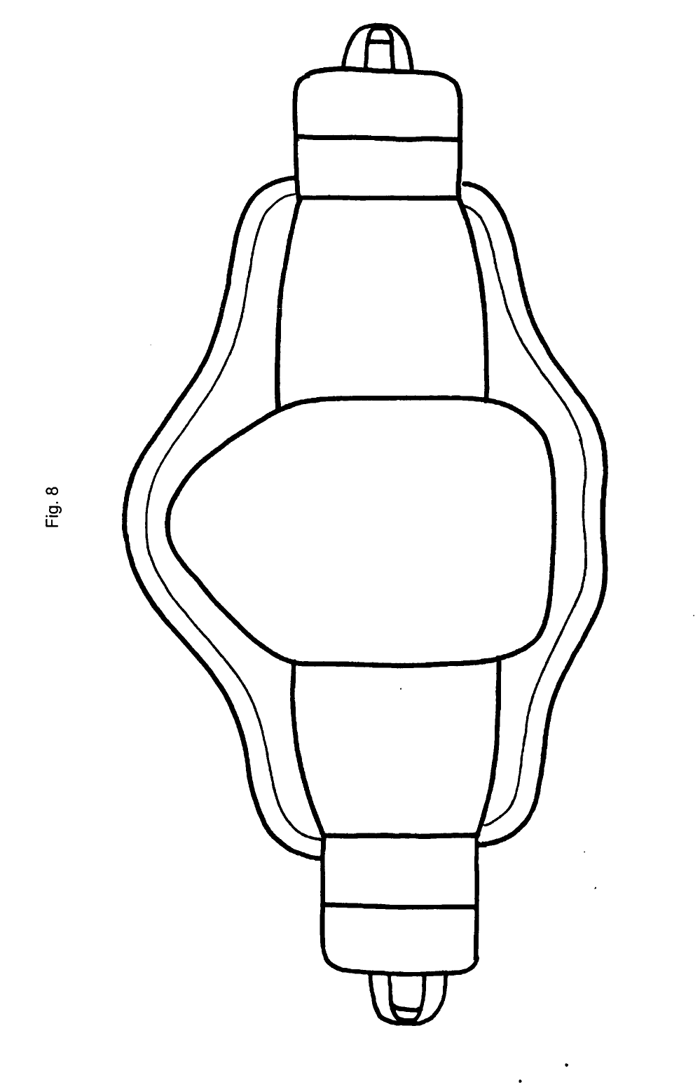

1. An orthosis comprising:
Lumbo Sacral Orthosis (apparatus) is comprised of the anterior and posterior rigid medical grade ABS heat moldable plastic. The anterior and posterior panels are lined with foam covered with a Velcro hook and loop fastener type material. These are affixed to the panels by a hook and loop fastener. The anterior and posterior panels are placed on the torso of the patient with an adjustable strap system consisting of elastic material with an internal plastic moldable insert, which is attached to the posterior panel via two brass oxide rivets. The strapping system is attached to the anterior panel via hook and loop material. This allows for the patient to apply desired tension. The secondary strapping system is affixed to the posterior panel via two brass oxide rivets attached to heat moldable plastic material. The anterior panel secondary strapping system is affixed to the panel via two stainless steel rivets. The anterior and posterior secondary strapping system is then connected via a buckle system. The C-Fold from the posterior section is hook and loop material and is able to remove any pressure from the posterior section and allows the patient to apply additional tension. The design of the LSO increases patient compliance due to the comfort and conforming nature of the liner and the plurality of straps provide the desired support.
